(19) United States
(12) Patent Application Publication
(10) Pub. No.: US 2015/0020688 A1
(43) Pub. Date: Jan. 22, 2015

(54) AIR FILTRATION DEVICE

(71) Applicants: Gary Lee Fairbank, JR., Royal Oak, MD (US); Dylan Ray Fairbank, Sherwood, MD (US)

(72) Inventors: Gary Lee Fairbank, JR., Royal Oak, MD (US); Dylan Ray Fairbank, Sherwood, MD (US)

(21) Appl. No.: 14/489,378

(22) Filed: Sep. 17, 2014

Related U.S. Application Data
(60) Provisional application No. 61/958,001, filed on Jul. 17, 2013.

(51) Int. Cl. A61L 9/14; B01D 46/00; A61L 9/20

(52) U.S. Cl. CPC A61L 9/14; A61L 9/20; B01D 46/0028
USPC 96/224; 96/227

(57) ABSTRACT

An air filtration device is provided. The air filtration device includes a high-efficiency particulate air filter negative air machine and a cold air fogger. The negative air machine includes an intake and an output for airflow. The cold air fogger may include a tank for including the fogging liquid and a delivery nozzle directed towards the output of the airflow of the negative air machine. Therefore, infected air may travel through the negative air machine and out of the output where the airflow is treated by the cold air fogger. The present invention may treat air within contaminated rooms.

### CROSS-REFERENCE TO RELATED APPLICATION

This application claims the benefit of priority of U.S. provisional application No. Jul. 17, 2013, filed 61/958,001, the contents of which are herein incorporated by reference.

### BACKGROUND OF THE INVENTION

The present invention relates to air filtration device and, more particularly, to a negative air machine combined with a cold air fogger.

Air disinfectants are typically chemical substances capable of disinfecting microorganisms suspended in the air. Although glycols are effective air disinfectants in controlled laboratory environments, it is more difficult to use them effectively in real-world environments because the disinfection of aft is sensitive to continuous action. Mold intrusions are usually treated by spraying with bleach or contained through demolition. Current mold remediation is timely and therefore costly. The same can be said about attempting to disinfect an area affected with airborne viral particles.

As can be seen, there is a need for an improved air filtration devices to treat infected indoor areas.

### SUMMARY OF THE INVENTION

In one aspect of the present invention, an air filtration device comprises: a negative air machine comprising an air filter within, wherein the negative air machine comprises an intake and an output for air; and a cold air fogger comprising a tank and a delivery nozzle, wherein the tank contains a mixture of hydrogen peroxide and silver ions and wherein the delivery nozzle is directed towards the output of the negative air machine.

These and other features, aspects and advantages of the present invention will become better understood with reference to the following drawings, description and claims.

### BRIEF DESCRIPTION OF THE DRAWINGS

FIG. 1 is a front perspective view of an embodiment of the present invention;

FIG. 2 is a back perspective view of an embodiment of the present invention;

FIG. 3 is an exploded view of an embodiment of the present invention;

FIG. 4 is an exploded view of the air dispersion assembly of FIG. 1;

FIG. 5 is a front view of an embodiment of the present invention; and

FIG. 6 is a side view of an embodiment of the present invention

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an air filtration device. The air filtration device includes a high-efficiency particulate air (HEPA) filter negative air machine and a cold air fogger. The negative air machine includes an intake and an output for airflow. The cold air fogger may include a tank for containing the fogging liquid with an adjustable electrostatic charge and a delivery nozzle directed towards the output of the airflow of the negative air machine. Therefore, infected air may travel through the negative air machine and out of the output where the airflow is treated by the cold air fogger. The present invention may treat air within any contaminated indoor areas and the present invention may easily disassemble and assemble within minutes.

The present invention includes a machine comprised of a combination of environmental systems which delivers laboratory quality indoor air. The machine removes fungal and viral particles from within the air as well salmonella, MERSA and other biological organisms with mitochondria and DNA. The machine utilizes a combination of a HEPA negative air machine, a dry fog comprised of hydrogen peroxide and silver ions with adjustable electrostatic charge as well as UV bacteria disinfection light to provide clean indoor air. The present invention can be set in an affected area and within a few hours the air will be clean and mold/bacterial/viral particles on surfaces and in the air are inert.

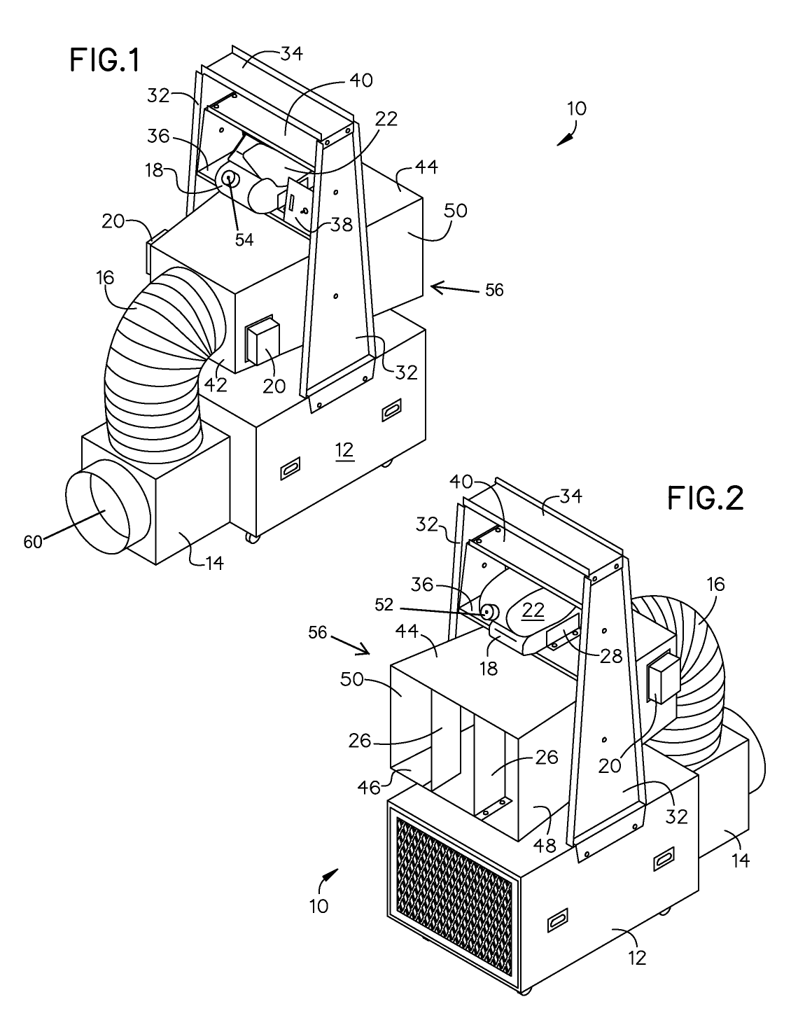

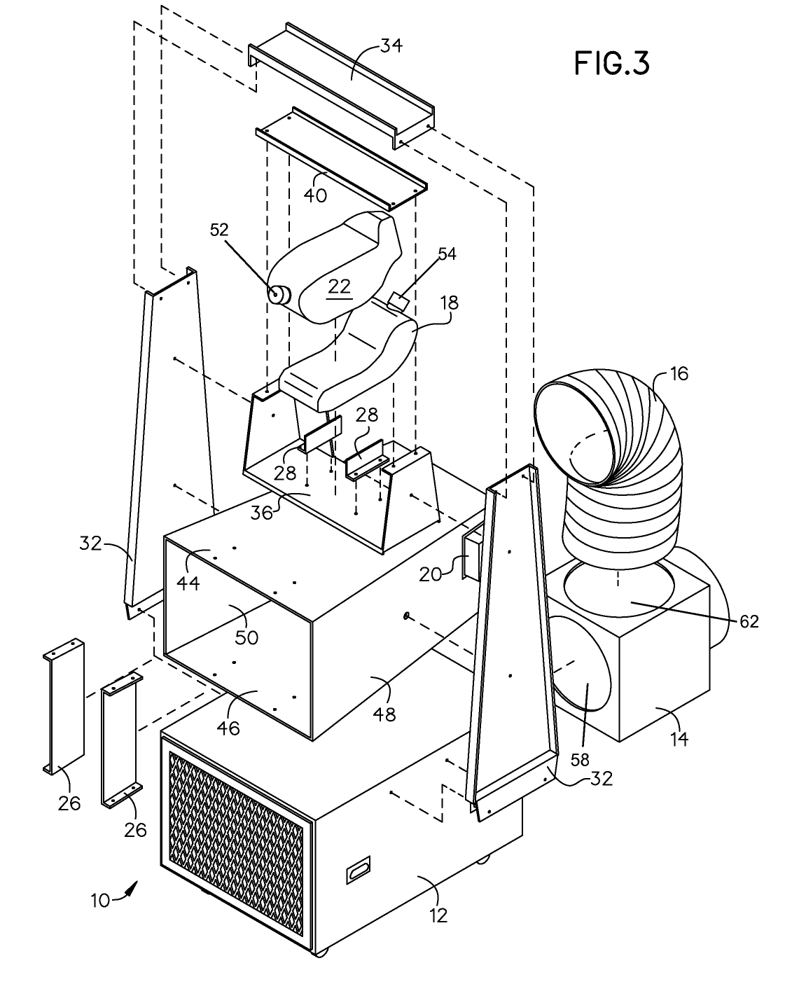

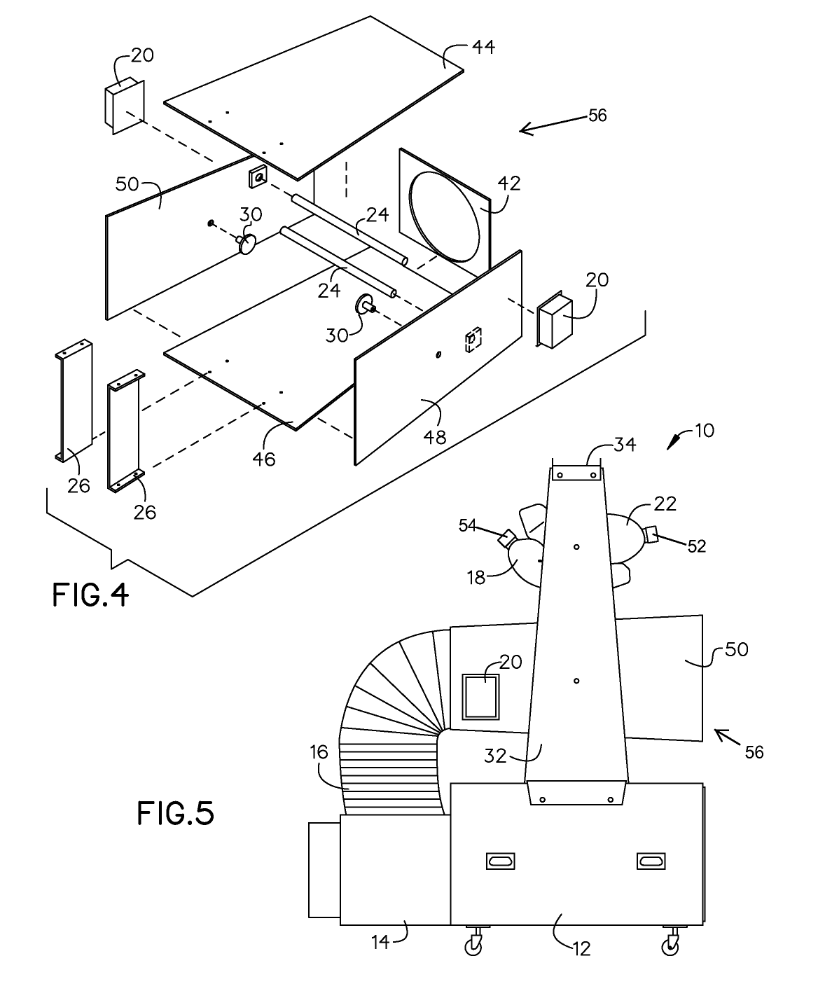

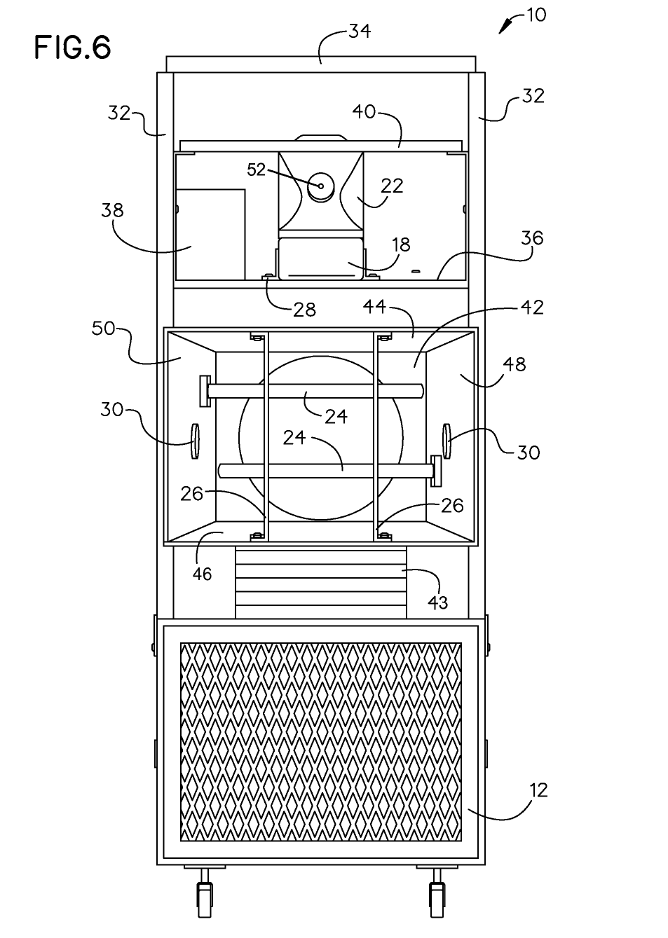

Referring to FIGS. 1 through 6, the present invention includes an air filtration device 10. The air filtration device 10 includes a combination of a negative air machine 12 and a cold air fogger 22. The negative air machine 12 may include an air filter, such as a high-efficiency particulate air (HEPA) filter, custom or high activated carbon filters for odor control within. The negative air machine 12 further includes an intake and an output for air. The HEPA negative air machine 12 is capable of moving a minimum of 2000 cubic feet per minute.

The cold air fogger 22 may include a tank 18 and a delivery nozzle 52. The tank 18 contains a mixture of hydrogen peroxide and silver ions and includes a cap 54 which may be removed to fill the tank 18 with the mixture. The cold air fogging machine 22 may include an adjustable droplet output size of about 10 to 40 microns and a dispersal rate of about 0.5 to 2 gallons per hour. An electric output is operable to charge the mixture, which amplifies the effects of the disinfecting fog. An electrostatic charge may be activated by a power source and may be adjustable for fine tuning the fogging system. The present invention may disperse ionized silver which causes induction of H²O² to mitochondria or bacteria. The cold air fogger 22 of the present invention dispenses the disinfecting fog towards the output of air from the negative air machine 12.

In certain embodiments, the present invention may include an air dispersion assembly 56. The air dispersion assembly 56 may be connected to the output of negative air machine 12 and may be positioned to blow air in an opposite direction of the intake of air of the negative air machine 12. The air dispersion assembly 56 may include an intake panel 42 include an opening, and a top panel 44, a bottom panel 46, a first side panel 48 and a second side panel 50 forming a channel within.

The present invention may further include a frame. The frame may mount the air dispersion assembly 56 above the negative air machine 12 and the cold air fogger 22 above the air dispersion assembly 56. As illustrated in the Figures, the frame may include side support beams 32 connected at the top by a top support beam 34. Below the top support beam 34 may be the middle shelf 40 and the sprayer shelf 36. The cold air fogger 18 may be secured in between the middle shelf 40 and the sprayer shelf 36 by supports 28. An adjustable electrostatic power source may run to the fogging fluid storage tank with an on/off switch delivered to the fogging mixture. Further, an on and off switch 38 may be secured to the sprayer shelf 36. The on and off switch 38 may be used to turn the air filtration device 10 on and off.

The air dispersion assembly 56 may be mounted to the frame below the sprayer shelf 36 and above the negative air machine 12. In certain embodiments, the air dispersion assembly 56 may be pivotally mounted to the supports 32 by swivel pins 30. A directional air flow panel 43 is mounted to the air dispersion assembly 56 in order to pivot the air dispersion assembly 56 and support the air dispersion assembly 56 in the pivoted direction.

The present invention may further include a vent box 14 and a hose 16. The vent box may include an intake opening 58, a delivery opening 62, and an exhaust opening 60. The intake opening 58 is connected to the output of the negative air machine and the hose 16 connects the delivery opening 62 to the intake panel 42 of air dispersion assembly 56. The layout described above achieves a separation of positive and negative air flow for the purpose of filtering out particulate including biological and viral contaminants. In certain embodiments, a 4 inch opening is located in between the hose 16 and the air dispersion assembly 56 to allow for an even greater degree of concentrated air flow which allows for removal of contagions in smaller, hard to reach areas.

The exhaust opening 60 of the vent box 14 may be used to utilize the negative air machine 12 alone. Therefore, one of the exhaust opening 60 and the delivery opening 62 are closed during operation of the air filtration device 10.

The inside of the air dispersion assembly 56 may include additional components. For example, at least one Ultra violet (UV) lamp 24 may be suspended within the air dispersion assembly 56. In certain embodiments two UV lamps 24 are installed inside of the air dispersion assembly 56. The UV lamps work in conjunction with the cold air fogger 22 to provide multiple levels of protection as these lights kill many types of viral/fungal contaminates.

Further, in certain embodiments the present invention may include a plurality of directional vents 26 mounted within the air dispersion assembly 56.

The present invention may further include a dehumidifier or a building dryer. The dehumidifier may be placed at one end of the present invention to quicken the disinfection process by aiding in the drying of the vapor dispelled by the cold air fogger.

The cold air fogger may spray a disinfection solution that contains a mixture of hydrogen peroxide (H2O2) and silver ions. When the solution comes into contact with any biological/viral pathogen the silver ion serves to punch a hole in the cellular membrane of the contaminant. The hydrogen peroxide then infiltrates the cell through this pathway and reacts with the processes within the mitochondria shutting down energy production within the contaminant and effectively rendering the pathogen dead. The electrostatic charge delivered to the fogging mixture attached to the nozzle of the fogger serves to enhance the ability of the silver ions to permeate cell membranes. After about 90 minutes the hydrogen peroxide reacts with atmospheric oxygen to form oxygen and water. Therefore, the entire process is clean, green and completely harmless. The HEPA negative air machine serves to filter the air of all harmful particulates as well as influencing the directionality of the disinfecting fog to wherever the user desires. The attached ultraviolet disinfection light also provides additional protection as it kills certain fungal/viral particles upon contact.

The machine is simple to operate. A user may simply place the machine in the center of the affected area, fill it with the appropriate amount of disinfection liquid, set it to operate for the appropriate time (both determined by the size of the area and level of disinfection required) and leave the room. Returning is possible in 2 hours as soon as the hydrogen peroxide oxygenates turning H²O² into H²O with a free oxygen molecule which will not raise oxygen levels in the area with the small amount of solution used in a room. At this time the indoor air is disinfected and the machine can be removed.

It should be understood, of course, that the foregoing relates to exemplary embodiments of the invention and that modifications may be made without departing from the spirit and scope of the invention as set forth in the following claims.

What is claimed is:

1. An air filtration device comprising:
a negative air machine comprising an air filter within, wherein the negative air machine comprises an intake and an output for air; and
a cold air fogger comprising a tank and a delivery nozzle, wherein the tank contains a mixture of hydrogen peroxide and silver ions and, wherein the delivery nozzle is directed towards the output of the negative air machine.

2. The air filtration device of claim 1, wherein the filter is a high-efficiency particulate air filter and comprises activated carbon.

3. The air filtration device of claim 1, further comprising an electric output operable to charge the mixture.

4. The air filtration device of claim 1, further comprising an air dispersion assembly connected to the output and positioned to blow air in the opposite direction of the intake air.

5. The air filtration device of claim 4, further comprising a vent box and a hose, wherein the vent box comprises an intake opening and a delivery opening, wherein the intake opening is connected to the output and the hose connects the delivery opening to the air dispersion assembly.

6. The air filtration device of claim 5, wherein the vent box further comprises an exhaust opening, wherein one of the exhaust opening and the delivery opening are closed during operation of the air filtration device.

7. The air filtration device of claim 4, further comprising a frame, wherein the air dispersion assembly is mounted to the frame above the negative air machine.

8. The air filtration device of claim 7, wherein the air dispersion assembly is pivotally mounted to the frame.

9. The air filtration device of claim 4, further comprising at least one Ultra violet lamp suspended within the air dispersion assembly.

10. The air filtration device of claim 4, further comprising a plurality of directional vents mounted within the air dispersion assembly. *The sleeve is for directional flow on air box and is not involved
